(19) United States
(12) Patent Application Publication
(10) Pub. No.: US 2004/0060125 A1
(43) Pub. Date: Apr. 1, 2004

(54) Oxidation dyeing composition for keratinous fibres comprising a 3,5-diamino-pyridine derivative and a particular thickening polymer

(76) Inventor: Marie-Pascale Audouset, Asnieres (FR)

Correspondence Address: FINNEGAN, HENDERSON, FARABOW, GARRETT & DUNNER, LLP, 1300 I STREET, NW, WASHINGTON, DC, 20005, US

(21) Appl. No.: 10/398,423

(22) PCT Filed: Mar. 21, 2001
(86) PCT No.: PCT/FR01/00846

(30) Foreign Application Priority Data
Apr. 12, 2000 (FR) 00/04721

(51) Int. Cl. A61K 7/13

(52) U.S. Cl. 8/405

(57) ABSTRACT

The invention concerns an oxidation dyeing composition for keratinous fibres, in particular human keratinous fibres such as hair, comprising, in a medium suitable for dyeing, at least a coupling agent selected among 3,5-diamino-pyridine derivatives and their addition salts with an acid, at least an oxidation base, and a particular thickening polymer. The invention also concerns dyeing methods and devices using said composition.

The present invention relates to a composition for the oxidation dyeing of keratin fibers, in particular of human keratin fibers such as the hair, comprising, in a medium that is suitable for dyeing, at least one coupler chosen from 3,5-diaminopyridine derivatives and the addition salts thereof with an acid, at least one oxidation dye precursor and at least one particular thickening polymer defined below.

It is known practice to dye keratin fibers, and in particular human hair, with dye compositions containing oxidation dye precursors, generally known as “oxidation bases”, in particular ortho- or para-phenylenediamines, ortho- or para-aminophenols and heterocyclic bases.

Oxidation dye precursors are compounds that are initially uncolored or only weakly colored and which develop their dyeing power on the hair in the presence of oxidizing agents, leading to the formation of colored compounds. The formation of these colored compounds results either from an oxidative condensation of the “oxidation bases” with themselves or from an oxidative condensation of the “oxidation bases” with coloration modifiers, or “couplers”, which are generally present in the dye compositions used in oxidation dyeing and are represented more particularly by meta-phenylenediamines, meta-aminophenols and meta-diphenols, and certain heterocyclic compounds.

The variety of molecules used, which consist on the one hand of the “oxidation bases” and on the other hand of the “couplers”, allows a very wide range of colors to be obtained.

The “permanent” coloration obtained by means of these oxidation dyes must moreover satisfy a certain number of requirements. Thus, it must allow shades to be obtained with the desired intensity and must show good resistance to external agents (light, bad weather, washing, permanent waving, perspiration and rubbing).

The dyes must also allow gray hair to be covered, and finally they must be as unselective as possible, i.e. they must produce the smallest possible color differences along a same length of keratin fiber, which may in fact be differently sensitized (i.e. damaged) between its end and its root.

Thus, oxidation dye compositions containing certain 3,5-diaminopyridine derivatives as coupler, in combination with oxidation bases conventionally used in oxidation dyeing, have already been proposed, especially in patents U.S. Pat. No. 4,473,375 or DE-3 132 885.

However, the colorations obtained using these compositions are not always sufficiently strong, chromatic or resistant to the various attacking factors to which the hair may be subjected.

The Applicant has now discovered that it is possible, entirely surprisingly and unexpectedly, to obtain novel oxidation dyes capable of giving chromatic, strong, esthetic, unselective colorations in varied shades, that show good resistance to the various attacking factors to which fibers may be subjected, by combining at least one coupler chosen from the 3,5-diaminopyridines of formula (I) defined below and the addition salts thereof with an acid, at least one oxidation dye precursor and at least one particular thickening polymer defined below.

This discovery forms the basis of the present invention.

A first subject of the invention is thus a composition for the oxidation dyeing of keratin fibers, and in particular of human keratin fibers such as the hair, comprising, in a medium that is suitable for dyeing:

(a) as coupler, at least one 3,5-diaminopyridine derivative of formula (I) below:

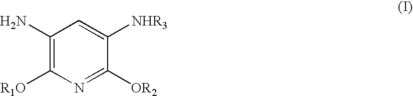

in which

R₁ and R₂, which may be identical or different, represent a C₁-C₄ alkyl, C₁-C₄ monohydroxyalkyl or C₂-C₄ polyhydroxyalkyl radical,

R₃ represents a hydrogen atom or a C₁-C₄ alkyl, C₁-C₄ monohydroxyalkyl or C₂-C₄ polyhydroxyalkyl radical, or one of the addition salts thereof with an acid; and

(b) at least one oxidation dye precursor, characterized in that it also comprises at least one thickening polymer chosen from the group consisting of:

(i) amphiphilic polymers comprising at least one fatty chain;

(ii) crosslinked acrylic acid homopolymers;

(iii) crosslinked copolymers of (meth)acrylic acid and of a (C₁-C₆)alkyl acrylate;

(iv) nonionic homopolymers and copolymers containing ethylenically unsaturated monomers of ester and/or amide type;

(v) crosslinked homopolymers of 2-acrylamido-2-methylpropanesulfonic acid and the partially or totally neutralized crosslinked acrylamide copolymers thereof;

(vi) ammonium acrylate homopolymers or copolymers of ammonium acrylate and of acrylamide;

(vii) nonionic guar gums;

(viii) biopolysaccharide gums of microbial origin and in particular scleroglucan gum or xanthan gum;

(ix) gums derived from plant exudates, and in particular gum arabic, ghatti gum, karaya gum, gum tragacanth, carrageenan gum, agar gum and carob gum;

(x) hydroxypropylcelluloses or carboxyalkylcelluloses;

(xi) pectins;

(xii) alginates.

The dye composition in accordance with the invention thus defined gives, after mixing with an oxidizing composition, chromatic, strong, esthetic colorations in varied shades, showing low selectivity and excellent properties of resistance not only to atmospheric agents such as light and bad weather, but also to perspiration and the various treatments to which the hair may be subjected.

Another subject of the invention relates to a ready-to-use composition for the oxidation dyeing of keratin fibers, which comprises, in a medium that is suitable for dyeing, at least one 3,5-diaminopyridine of formula (I), at least one oxidation dye precursor, at least one thickening polymer defined above and at least one oxidizing agent, with the exclusion of 2,6-dimethoxy-3,5-diaminopyridine combined with the methacrylic acid/ethyl acrylate/stearyl methacrylate oxyethylenated terpolymer with a uricase-containing enzymatic oxidizing system.

For the purposes of the present invention, the expression “ready-to-use composition” means any composition intended to be applied immediately to keratin fibers.

The invention is also directed toward a process for the oxidation dyeing of keratin fibers, and in particular of human keratin fibers such as the hair, which consists in applying to the fibers a dye composition containing, in a medium that is suitable for dyeing, at least one 3,5-diaminopyridine of formula (I) and at least one oxidation dye precursor, the color being developed at alkaline, neutral or acidic pH with the aid of an oxidizing composition that is mixed with the dye composition just at the time of use, or that is applied sequentially without intermediate rinsing, at least one thickening polymer defined according to-the invention being present in the dye composition and/or oxidizing composition.

A subject of the invention is also multi-compartment dyeing devices or “kits” for the oxidation dyeing of keratin fibers, in particular of human keratin fibers such as the hair.

Such devices comprise a first compartment containing at least one 3,5-diaminopyridine of formula (I) and at least one oxidation dye precursor and a second compartment containing an oxidizing agent, at least one thickening polymer defined according to the invention being present in the first compartment and/or in the second compartment.

Another multi-compartment dyeing device comprises at least one compartment containing at least one 3,5-diaminopyridine of formula (I) and at least one oxidation dye precursor, at least one compartment containing at least one thickening polymer defined according to the invention, and at least one other compartment containing at least one oxidizing agent.

However, other characteristics, aspects, objects and advantages of the invention will emerge even more clearly on reading the description and the examples that follow.

Among the 3,5-diaminopyridine derivatives of formula (I) in accordance with the invention, mention may be made of 2,6-dimethoxy-3,5-diaminopyridine, 2,6-diethoxy-3,5-diaminopyridine and 2,6-di(β-hydroxyethyloxy)-3,5-diaminopyridine, and the addition salts thereof with an acid.

According to the invention, the dye composition preferably contains 2,6-dimethoxy-3,5-diaminopyridine, and/or at least one of the addition salts thereof with an acid.

The 3,5-diaminopyridine derivative(s) of formula (I) that may be used in the dye composition in accordance with the invention preferably represent(s) from 0.0001% to 10% by weight approximately relative to the total weight of the dye composition, and even more preferably from 0.005% to 5% by weight approximately relative to this weight.

The dye composition in accordance with the invention contains at least one oxidation dye precursor or oxidation base.

The nature of these oxidation bases is not critical.

They may especially be chosen from para-phenylenediamines, double bases, para-aminophenols, ortho-aminophenols and heterocyclic oxidation bases.

Among the para-phenylenediamines that may be used as oxidation base in the dye compositions in accordance with the invention, mention may be made especially of the compounds of formula (II) below, and the addition salts thereof with an acid:

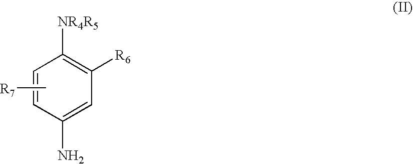

in which:

R₄ represents a hydrogen atom, a C₁-C₄ alkyl radical, a C₁-C₄ monohydroxyalkyl radical, a C₂-C₄ poly-hydroxyalkyl radical, a (C₁-C₄)alkoxy(C₁-C₄)alkyl radical, or a C₁-C₄ alkyl radical substituted with a nitrogenous group, a phenyl group or a 4′-aminophenyl group;

R₅ represents a hydrogen atom, a C₁-C₄ alkyl radical, a C₁-C₄ monohydroxyalkyl radical, a C₂-C₄ poly-hydroxyalkyl radical, a (C₁-C₄)alkoxy(C₁-C₄)alkyl radical or a C₁-C₄ alkyl radical substituted with a nitrogenous group;

R₆ represents a hydrogen atom, a halogen atom such as a chlorine, bromine, iodine or fluorine atom, a C₁-C₄ alkyl radical, a C₁-C₄ monohydroxyalkyl radical, a C₁-C₄ hydroxyalkoxy radical, an acetylamino(C₁-C₄)alkoxy radical, a C₁-C₄ mesylaminoalkoxy radical or a carbamoylamino(C₁-C₄)alkoxy radical,

R₇ represents a hydrogen or halogen atom or a C₁-C₄ alkyl radical.

Among the nitrogenous groups of formula (II) 15. above, mention may be made especially of amino, mono(C₁-C₄)alkylamino, di(C₁-C₄)alkylamino, tri(C₁-C₄)alkylamino, monohydroxy(C₁-C₄)alkylamino, imidazolinium and ammonium radicals.

Among the para-phenylenediamines of formula (II) above, mention may be made more particularly of para-phenylenediamine, para-tolylenediamine, 2-chloro-para-phenylene-diamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-p-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N,N-(ethyl-β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxy-ethyloxy-para-phenylenediamine, 2-β-acetylamino-ethyloxy-para-phenylenediamine, N-(β-methoxyethyl)-para-phenylenediamine and 2-methyl-1-N-(β-hydroxyethyl)-para-phenylenediamine, and the addition salts thereof with an acid.

Among the para-phenylenediamines of formula (II) above, para-phenylenediamine, para-tolylenediamine, 2-isopropyl-para-phenylenediamine, 2-β-hydroxyethyl-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine and 2-chloro-para-phenylenediamine, and the addition salts thereof with an acid, are most particularly preferred.

According to the invention, the term “double bases” means compounds comprising at least two aromatic nuclei bearing amino and/or hydroxyl groups.

Among the double bases that may be used as oxidation bases in the dye composition in accordance with the invention, mention may be made in particular of the compounds corresponding to formula (III) below, and the addition salts thereof with an acid:

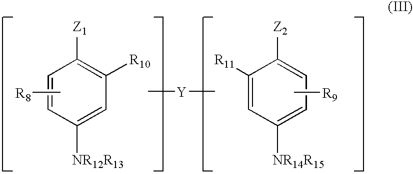

in which:

Z₁ and Z₂, which may be identical or different, represent a hydroxyl or —NH₂ radical which may be substituted with a C₁-C₄ alkyl radical or with a linker arm Y;

the linker arm Y represents a linear or branched alkylene chain containing from 1 to 14 carbon atoms, which may be interrupted or terminated with one or more nitrogenous groups and/or one or more hetero atoms such as oxygen, sulfur or nitrogen atoms, and optionally substituted with one or more hydroxyl or C₁-C₆ alkoxy radicals;

R₈ and R₉ represent a hydrogen or halogen atom, a C₁-C₄ alkyl radical, a C₁-C₄ monohydroxyalkyl radical, a C₂-C₄ polyhydroxyalkyl radical, a C₁-C₄ aminoalkyl radical or a linker arm Y;

R₁₀, R₁₁, R₁₂, R₁₃, R₁₄ and R₁₅, which may be identical or different, represent a hydrogen atom, a linker arm Y or a C₁-C₄ alkyl radical; it being understood that the compounds of formula (III) contain only one linker arm Y per molecule.

Among the nitrogenous groups [lacuna] of formula (III) above, mention may be made especially of amino, mono(C₁-C₄)alkylamino, di(C₁-C₄)alkylamino, tri(C₁-C₄)alkylamino, monohydroxy(C₁-C₄)alkylamino, imidazolinium and ammonium radicals.

Among the double bases of formula (III) above, mention may be made more particularly of N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)-tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)-ethylenediamine and 1,8-bis(2,5-diaminophenoxy)-3,5-dioxaoctane, and the addition salts thereof with an acid.

Among these double bases of formula (III), N,N′-bis(β-hydroxyethyl)-N,N′-bis(4′-aminophenyl)-1,3-diaminopropanol and 1,8-bis(2,5-diaminophenoxy)-3,5-dioxaoctane, or one of the addition salts thereof with an acid, are particularly preferred.

Among the para-aminophenols that may be used as oxidation bases in the dye compositions in accordance with the invention, mention may be made especially of the compounds corresponding to formula (IV) below, and the addition salts thereof with an acid:

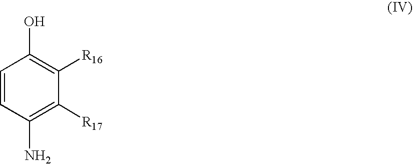

in which:

R₁₆ represents a hydrogen or halogen atom or a C₁-C₄ alkyl, C₁-C₄ monohydroxyalkyl, (C₁-C₄)alkoxy(C₁-C₄)-alkyl, C₁-C₄ aminoalkyl or hydroxy(C₁-C₄)-alkylamino (C₁-C₄) alkyl radical,

R₁₇ represents a hydrogen or halogen atom or a Cₗ-C₄ alkyl, C₁-C₄ monohydroxyalkyl, C₂-C₄ polyhydroxyalkyl, C₁-C₄ aminoalkyl, C₁-C₄ cyanoalkyl or (C₁-C₄)alkoxy(C₁-C₄)alkyl radical, it being understood that at least one of the radicals R₁₆ or R₁₇ represents a hydrogen atom.

Among the para-aminophenols of formula (IV) above, mention may be made more particularly of para-aminophenol, 4-amino-3-methylphenol, 4-amino-3-fluoro-phenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol and 4-amino-2-fluorophenol, and the addition salts thereof with an acid.

Among the ortho-aminophenols that can be used as oxidation bases in the dye compositions in accordance with the invention, mention may be made more particularly of 2-aminophenol, 2-amino-5-methylphenol, 2-amino-6-methylphenol and 5-acetamido-2-aminophenol, and the addition salts thereof with an acid.

Among the heterocyclic bases that may be used as oxidation bases in the dye compositions in accordance with the invention, mention may be made more particularly of pyridine derivatives, pyrimidine derivatives and pyrazole derivatives, and the addition salts thereof with an acid.

Among the pyridine derivatives, mention may be made more particularly of the compounds described, for example, in patents GB 1 026 978 and GB 1 153 196, for instance 2,5-diaminopyridine, 2-(4-methoxyphenyl)amino-3-aminopyridine, 2,3-diamino-6-methoxypyridine, 2-(β-methoxyethyl)amino-3-amino-6-methoxypyridine and 3,4-diaminopyridine, and the addition salts thereof with an acid.

Among the pyrimidine derivatives, mention may be made more particularly of the compounds described, for example, in German patent DE 2 359 399 or Japanese patents JP 88-169 571 and JP 91-10659 or patent application WO 96/15765, for instance 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine and 2,5,6-triaminopyrimidine, and pyrazolopyrimidine derivatives such as those mentioned in patent application FR-A-2 750 048, and among which mention may be made of pyrazolo[1,5-a]pyrimidine-3,7-diamine; 2,5-dimethylpyrazolo[1,5-a]pyrimidine-3,7-diamine; pyrazolo[1,5-a]pyrimidine-3,5-diamine; 2,7-dimethylpyrazolo[1,5-a]pyrimidine-3,5-diamine; 3-aminopyrazolo[1,5-a]pyrimidin-7-ol; 3-aminopyrazolo[1,5-a]pyrimidin-5-ol; 2-(3-aminopyrazolo[1,5-a]pyrimidin-7-ylamino)ethanol; 2-(7-aminopyrazolo[1,5-a]pyrimidin-3-ylamino)ethanol, 2-[(3-aminopyrazolo[1,5-a]pyrimidin-7-yl)-(2-hydroxyethyl)amino]ethanol, 2-[(7-amino-pyrazolo[1,5-a]pyrimidin-3-yl)-(2-hydroxyethyl)amino]-ethanol, 5,6-dimethylpyrazolo[1,5-a)pyrimidine3,7-diamine, 2,6-dimethylpyrazolo[1,5-a]pyrimidine-3,7diamine, 2,5,N7,N7-tetramethylpyrazolo[1,5-a]-pyrimidine-3,7-diamine, 3-amino-5-methyl-7-imidazolylpropylaminopyrazolo[1,5-a]pyrimidine, the tautomeric forms thereof, when a tautomeric equilibrium exists, and the addition salts thereof with an acid.

Among the pyrazole derivatives, mention may be made more particularly of the compounds described in patents DE 3 843 892, DE 4 133 957 and patent applications WO 94/08969, WO 94/08970, FR-A-2 733 749 and DE 195 43 988, for instance 4,5-diamino-1-methyl-pyrazole, 3,4-diaminopyrazole, 4,5-diamino-1-(4′-chlorobenzyl)pyrazole, 4,5-diamino-1,3-dimethyl-pyrazole, 4,5-diamino-3-methyl-1-phenylpyrazole, 4,5-diamino-1-methyl-3-phenylpyrazole, 4-amino-1,3-di-methyl-5-hydrazinopyrazole, 1-benzyl-4,5-diamino-3-methylpyrazole, 4,5-diamino-3-tert-butyl-1-methyl-pyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-amino-ethyl)amino-1,3-dimethylpyrazole, 3,4,5-triamino-pyrazole, 1-methyl-3,4,5-triaminopyrazole, 3,5-diamino-1-methyl-4-methylaminopyrazole and 3,5-diamino-4-(β-hydroxyethyl)amino-1-methylpyrazole, and the addition salts thereof with an acid.

According to one preferred embodiment of the invention, the oxidation base(s) is (are) chosen from. para-phenylenediamines.

The oxidation base(s) preferably represent(s) from 0.0005% to 12% by weight approximately relative to the total weight of the dye composition in accordance with the invention, and even more preferably from 0.005% to 8% by weight approximately relative to this weight.

Thickening Polymers According to the Invention

(i) The amphiphilic thickening polymers comprising at least one fatty chain according to the invention are preferably chosen from those of anionic, nonionic or cationic type.

Amphiphilic Thickening Polymers of Anionic Type Comprising at Least One Fatty Chain

Among these, mention may be made of:

(I) those comprising at least one hydrophilic unit and at least one fatty-chain allyl ether unit, more particularly those in which the hydrophilic unit consists of an ethylenic unsaturated anionic monomer, even more particularly a vinylcarboxylic acid and most particularly an acrylic acid, a methacrylic acid or mixtures thereof, the fatty-chain allyl ether unit corresponding to the monomer of formula (V) below:

CH₂═CR′CH₂OBₙR (V)

in which R′ denotes H or CH₃, B denotes an ethylenoxy radical, n is zero or denotes an integer ranging from 1 to 100, R denotes a hydrocarbon-based radical chosen from alkyl, arylalkyl, aryl, alkylaryl and cycloalkyl radicals, containing 8 to 30 carbon atoms, preferably 10 to 24 carbon atoms and even more particularly from 12 to 18 carbon atoms. A unit of formula (I) that is more particularly preferred is a unit in which R′ denotes H, n is equal to 10 and R denotes a stearyl (C₁₈) radical.

Anionic amphiphilic polymers of this type are described and prepared, according to an emulsion polymerization process, in patent EP-0 216 479.

Among these fatty-chain anionic thickening polymers, those that are particularly preferred according to the invention are polymers formed from 20% to 60% by weight of acrylic acid and/or of methacrylic acid, from 5% to 60% by weight of lower alkyl (meth)acrylates, from 2% to 50% by weight of fatty-chain allyl ether of formula (I), and from 0% to 1% by weight of a crosslinking agent that is a well-known copolymerizable unsaturated polyethylenic monomer, for instance diallyl phthalate, allyl (meth)acrylate, divinylbenzene, (poly)ethylene glycol dimethacrylate or methylenebisacrylamide.

Among the latter polymers, those most particularly preferred are crosslinked terpolymers of methacrylic acid, of ethyl acrylate and of polyethylene glycol (10 EO) stearyl ether (Steareth-10), especially those sold by the company Allied Colloids under the names Salcare SC 80 and Salcare SC 90, which are aqueous 30% emulsions of a crosslinked terpolymer of methacrylic acid, of ethyl acrylate and of steareth-10 allyl ether (40/50/10).

(II) those comprising at least one hydrophilic unit of unsaturated olefinic carboxylic acid type, and at least one hydrophobic unit of (C₁₀-C₃₀)alkyl ester of unsaturated carboxylic acid type.

Preferably, these polymers are chosen from those in which the hydrophilic unit of unsaturated olefinic carboxylic acid type corresponds to the monomer of formula (VI) below:

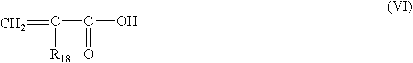

in which R₁₈ denotes H or CH₃ or C₂H₅, that is to say acrylic acid, methacrylic acid or ethacrylic acid units, and in which the hydrophobic unit of (C₁₀-C₃₀)alkyl ester of unsaturated carboxylic acid type corresponds to the monomer of formula (VII) below:

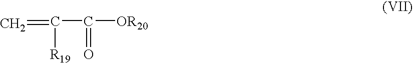

in which R₁₉ denotes H or CH₃ or C₂H₅ (that is to say acrylate, methacrylate or ethacrylate units) and preferably H (acrylate units) or CH₃ (methacrylate units), R₂₀ denoting a C₁₀-C₃₀ and preferably C₁₂-C₂₂ alkyl radical.

(C₁₀-C₃₀) alkyl esters of unsaturated carboxylic acids in accordance with the invention include, for example, lauryl acrylate, stearyl acrylate, decyl acrylate, isodecyl acrylate and dodecyl acrylate, and the corresponding methacrylates, lauryl methacrylate, stearyl methacrylate, decyl methacrylate, isodecyl methacrylate and dodecyl methacrylate.

Anionic polymers of this type are described and prepared, for example, according to U.S. Pat. Nos. 3,915,921 and 4,509,949.

Among the fatty-chain anionic thickening polymers of this type that will be used more particularly are polymers formed from a monomer mixture comprising:

(i) essentially acrylic acid,

(ii) an ester of formula (VII) described above in which R₁₉ denotes H or CH₃, R₂₀ denoting an alkyl radical containing from 12 to 22 carbon atoms,

(iii) and a crosslinking agent, which is a well-known copolymerizable polyethylenic unsaturated monomer, for instance diallyl phthalate, allyl (meth)acrylate, divinylbenzene, (poly)ethylene glycol dimethacrylate or methylenebisacrylamide.

Among the fatty-chain anionic thickening polymers of this type that will be used more particularly are those consisting of from 95% to 60% by weight of acrylic acid (hydrophilic unit), 4% to 40% by weight of C₁₀-C₃₀ alkyl acrylate (hydrophobic unit) and 0% to 6% by weight of crosslinking polymerizable monomer, or alternatively those consisting of from 98% to 96% by weight of acrylic acid (hydrophilic unit), 1% to 4% by weight of C₁₀-C₃₀ alkyl acrylate (hydrophobic unit) and 0.1% to 0.6% by weight of crosslinking polymerizable monomer such as those described above.

Among said above polymers, those most particularly preferred according to the present invention are the products sold by the company Goodrich under the trade names Pemulen TR1, Pemulen TR2 and Carbopol 1382, and even more preferentially Pemulen TR1, and the product sold by the company S.E.P.P.I.C under the name Coatex SX.

(III) maleic anhydride/C₃₀-C₃₈ α-olefin/alkyl maleate terpolymers, such as the product (maleic anhydride/C₃₀-C₃₈ α-olefin/isopropyl maleate copolymer) sold under the name Performa V 1608 by the company Newphase Technologies.

(IV) acrylic terpolymers comprising:

(a) about 20% to 70% by weight of a carboxylic acid containing α,β-monoethylenic unsaturation,

(b) about 20% to 80% by weight of a non-surfactant monomer containing α,β-monoethylenic unsaturation other than (a),

(c) about 0.5% to 60% by weight of a nonionic monourethane that is the product of reaction of a monohydric surfactant with a monoisocyanate containing. monoethylenic unsaturation,

such as those described in patent application EP-A-0 173 109 and more particularly the terpolymer described in example 3, namely a methacrylic acid/methyl acrylate/behenyl dimethyl-meta-isopropenylbenzyisocyanate ethoxylated (40 EO) terpolymer, as an aqueous 25% dispersion.

Fatty-Chain Amphiphilic Thickening Polymers of Nonionic Type

Among these, the ones that are preferably chosen are:

(1) celluloses modified with groups comprising at least one fatty chain;

examples that may be mentioned include:

hydroxyethylcelluloses modified with groups comprising at least one fatty chain, such as alkyl, arylalkyl or alkylaryl groups, or mixtures thereof, and in which the alkyl groups are preferably C₈-C₂₂, for instance the product Natrosol Plus Grade 330 CS (C₁₆ alkyls) sold by the company Aqualon, or the product Bermocoll EHM 100 sold by the company Berol Nobel.

those modified with alkylphenyl polyalkylene glycol ether groups, such as the product Amercell Polymer HM-1500 (nonylphenyl polyethylene glycol (15) ether) sold by the company Amerchol.

(2) hydroxypropyl guars modified with groups comprising at least one fatty chain, such as the product Esaflor HM 22 (C₂₂ alkyl chain) sold by the company Lamberti, and the products RE210-18 (C₁₄ alkyl chain) and RE205-1 (C₂₀ alkyl chain) sold by the company Rhone-Poulenc.

(3) copolymers of vinylpyrrolidone and of fatty-chain hydrophobic monomers; -examples

that may be mentioned include:

the products Antaron V216 or Ganex V216. (vinylpyrrolidone/hexadecene copolymer) sold by the company I.S.P.

the products Antaron V220 or Ganex V220 (vinylpyrrolidone/eicosene copolymer) sold by the company I.S.P.

(4) copolymers of C₁-C₆ alkyl methacrylates or acrylates and of amphiphilic monomers comprising at least one fatty chain, such as, for example, the oxyethylenated methyl acrylate/stearyl acrylate copolymer sold by the company Goldschmidt under the name Antil 208.

(5) copolymers of hydrophilic methacrylates or acrylates and of hydrophobic monomers comprising at least one fatty chain, such as, for example, the polyethylene glycol methacrylate/lauryl methacrylate copolymer.

(6) polyurethane polyethers comprising in their chain both hydrophilic blocks usually of polyoxyethylenated nature and hydrophobic blocks that may be aliphatic sequences alone and/or cycloaliphatic and/or aromatic sequences.

(7) polymers with an aminoplast ether skeleton containing at least one fatty chain, such as the Pure Thix compounds sold by the company Sud-Chemie.

Preferably, the polyurethane polyethers comprise at least two hydrocarbon-based lipophilic chains containing from 6 to 30 carbon atoms, separated by a hydrophilic block, the hydrocarbon-based chains possibly being pendent chains, or chains at the end of the hydrophilic block. In particular, it is possible for one or more pendent chains to be included. In addition, the polymer may comprise a hydrocarbon-based chain at one end or at both ends of a hydrophilic block.

The polyurethane polyethers may be multiblock, in particular in triblock form. The hydrophobic blocks may be at each end of the chain (for example: triblock copolymer with a hydrophilic central block) or distributed both at the ends and in the chain (for example: multiblock copolymer). These same polymers may also be graft polymers or starburst polymers.

The fatty-chain nonionic polyurethane polyethers may be triblock copolymers in which the hydrophilic block is a polyoxyethylenated chain comprising from 50 to 1 000 oxyethylene groups. The nonionic polyurethane polyethers comprise a urethane linkage between the hydrophilic blocks, whence arises the name.

By extension, also included among the fatty-chain nonionic polyurethane polyethers are those in which the hydrophilic blocks are linked to the lipophilic blocks via other chemical bonds.

As examples of fatty-chain nonionic polyurethane polyethers that may be used in the invention, mention may also be made of Rheolate 205 containing a urea function, sold by the company Rheox, or the Rheolates 208, 204 or 212, and also Acrysol RM 184, Aculyn 44 and Aculyn 46 from the company Rohm & Haas [Aculyn 46 is a polycondensate of polyethylene glycol containing 150 or 180 mol of ethylene oxide, of stearyl alcohol and of methylenebis(4-cyclohexyl isocyanate) (SMDI), at 15% by weight in a matrix of maltodextrin (4%) and water (81%); Aculyn 44 is a polycondensate of polyethylene glycol containing 150 or 180 mol of ethylene oxide, of decyl alcohol and of methylenebis(4-cyclohexyl-isocyanate) (SMDI), at 35% by weight in a mixture of propylene glycol (39%) and water (26%)].

Mention may also be made of the product Elfacos T210 containing a C₁₂₋₁₄ alkyl chain, and the product Elfacos T212 containing a C₁₈ alkyl chain, from Akzo.

The product DW 1206B from Rohm & Haas containing a C₂₀ alkyl chain and a urethane linkage, sold at a solids content of 20% in water, may also be used.

It is also possible to use solutions or dispersions of these polymers, especially in water or in aqueous-alcoholic medium. Examples of such polymers that may be mentioned are Rheolate 255, Rheolate 278 and Rheolate 244 sold by the company Rheox. The products DW 1206F and DW 1206J sold by the company Rohm & Haas may also be used.

The polyurethane polyethers that may be used according to the invention are in particular those described in the article by G. Fonnum, J. Bakke and Fk. Hansen—Colloid Polym. Sci 271, 380-389 (1993).

Fatty-Chain Amphiphilic Thickening Polymers of Cationic Type

Among these polymers, the ones that are used in the present invention are preferably chosen from quaternized cellulose derivatives and polyacrylates containing acyclic amino side groups.

The quaternized cellulose derivatives are, in particular,

quaternized celluloses modified with groups comprising at least one fatty chain, such as alkyl, arylalkyl or alkylaryl groups containing at least 8 carbon atoms, or mixtures thereof.

quaternized hydroxyethylcelluloses modified with groups comprising at least one fatty chain, such as alkyl, arylalkyl or alkylaryl groups containing at least 8 carbon atoms, or mixtures thereof.

The alkyl radicals borne by the above quaternized celluloses or hydroxyethylcelluloses preferably contain from 8 to 30 carbon atoms. The aryl radicals preferably denote phenyl, benzyl, naphthyl or anthryl groups.

Examples of quaternized alkylhydroxyethyl-celluloses containing C₈-C₃₀ fatty chains that may be mentioned include the products Quatrisoft LM 200, Quatrisoft LM-X 529-18-A, Quatrisoft LM-X 529-18B (C₁₂ alkyl) and Quatrisoft LM-X 529-8 (C₁₈ alkyl) sold by the company Amerchol, and the products Crodacel QM, Crodacel QL (C₁₂ alkyl) and Crodacel QS (C₁₈ alkyl) sold by the company Croda.

The polyacrylates containing quaternized or nonquaternized amino side groups contain, for example, hydrophobic groups of the type such as Steareth 20 (polyoxyethylenated(20) stearyl alcohol).

Examples of polyacrylates containing amino side chains that may be mentioned include the polymers 8781-121B and 9492-103 sold by the company National Starch.

In the oxidation dye composition according to the invention, it is preferred to use a fatty-chain amphiphilic thickening polymer of nonionic type.

(ii) Among the crosslinked acrylic acid homopolymers that may be mentioned are those crosslinked with an allylic alcohol ether of the sugar series, such as, for example, the products sold under the names Carbopol 980, 981, 954, 2984 and 5984 by the company Goodrich or the products sold under the names Synthalen M and Synthalen K by the company 3 VSA.

(iii) Among the crosslinked copolymers of (meth)acrylic acid and of C₁-C₆ alkyl acrylate that may be mentioned are the product sold under the name Viscoatex 538C by the company Coatex, which is a crosslinked copolymer of methacrylic acid and of ethyl acrylate as an aqueous dispersion containing 38% active material, or the product sold under the name Aculyn 33 by the company Rohm & Haas, which is a crosslinked copolymer of acrylic acid and of ethyl acrylate as an aqueous dispersion containing 28% active material.

(iv) Among the nonionic homopolymers or copolymers containing ethylenically unsaturated monomers of ester and/or amide type, mention may be made of the products sold under the names: Cyanamer P250 by the company Cytec (polyacrylamide); PMMA MBX-8C by the company US Cosmetics (methyl methacrylate/ethylene glycol dimethacrylate copolymer); Acryloid B66 by the company Rohm & Haas (butyl methacrylate/methyl methacrylate copolymer); BPA 500 by the company Kobo (polymethyl methacrylate).

(v) Among the crosslinked 2-acrylamido-2-methylpropanesulfonic acid homopolymers that may be mentioned are those described in patent application EP-A-0 815 828 (which forms an integral part of the content of the description). Among the 2-acrylamido-2-methylpropanesulfonic acid crosslinked copolymers that are partially or totally neutralized (with a base such as sodium hydroxide, potassium hydroxide or an amine), mention may be made in particular of the product described in example 1 of document EP-A-503 853 (which forms an integral part of the content of the description).

(vi) Among the ammonium acrylate homopolymers that may be mentioned is the product sold under the name Microsap PAS 5193 by the company Hoechst.

Among the copolymers of ammonium acrylate and of acrylamide that may be mentioned is the product sold under the name Bozepol C Nouveau or the product PAS 5193 sold by the company Hoechst (which are described and prepared in documents FR-2 416 723, U.S. Pat. No. 2,798,053 and U.S. Pat. No. 2,923,692).

(vii) The nonionic guar gums can be modified or unmodified. The unmodified guar gums are, for-example, the products sold under the name Vidogum GH 175 by the company Unipectine and under the names Meypro-Guar 50 and Jaguar C by the company Meyhall.

The modified nonionic guar gums are especially modified with C₁-C₆ hydroxyalkyl groups.

Among the hydroxyalkyl groups that may be mentioned, for example, are hydroxymethyl, hydroxyethyl, hydroxypropyl and hydroxybutyl groups. These guar gums are well known in the prior art and can be prepared, for example, by reacting the corresponding alkene oxides such as, for example, propylene oxides, with the guar gum so as to obtain a guar gum modified with hydroxypropyl groups.

The degree of hydroxyalkylation, which corresponds to the number of alkylene oxide molecules consumed by the number of free hydroxyl functions present on the guar gum, preferably ranges from 0.4 to 1.2.

Such nonionic guar gums optionally modified with hydroxyalkyl groups are sold, for example, under the trade names Jaguar HP8, Jaguar HP60 and Jaguar HP120, Jaguar DC 293 and Jaguar HP 105 by the company Rhône-Poulenc (Meyhall) or under the name Galactasol 4H4FD2 by the company Aqualon.

(viii) The biopolysaccharide gums of microbial origin, and in particular scleroglucan gum or xanthan gum, the gums derived from plant exudates such as gum arabic, ghatti gum, karaya gum, gum tragacanth, carrageenan gum, agar gum and carob gum, the hydroxypropylcelluloses and carboxyalkylcelluloses, pectins and alginates are well known to those skilled in the art and are described especially in the book by Robert L. Davidson entitled “Handbook of Water Soluble Gums and Resins” published by the McGraw Hill Book Company (1980).

Among these gums, the scleroglucans more particularly used according to the present invention are represented by the products sold under the name Actigum CS by the company Sanofi Bio Industries and in particular Actigum CS 11, and under the name Amigel by the company Alban Muller International. Other scleroglucans, such as the one treated with glyoxal in French patent application 2 633 940, can also be used.

The xanthans more particularly used according to the present invention are represented by the products sold under the names Keltrol, Keltrol T, Keltrol T F, Keltrol B T, Keltrol R D and Keltrol C G by the company Nutrasweet Kelco, or under the names Rhodicare S and Rhodicare H by the company Rhodia Chimie.

(x) Among the hydroxypropylcelluloses that may be mentioned are the products sold under the names Klucel E F, Klucel H, Klucel L H F, Klucel M F and Klucel G by the company Aqualon.

Among the carboxyalkylcelluloses preferably used is carboxymethylcellulose, for which mention may be made of the products sold under the names Blanose 7M8/SF, Blanose Raffinée 7M, Blanose 7LF, Blanose 7MF, Blanose 9M31F, Blanose 12M31XP, Blanose 12M31P, Blanose 9M31XF, Blanose 7H, Blanose 7M31 and Blanose 7H3SXF by the company Aqualon, or Aquasorb A500 and Ambergum 1221 by the company Hercules, or Cellogen HP810A and Cellogen HP6HS9 by the company Montello, or Primellose by the company Avebe.

The thickening polymer(s) thus defined, which may be used in the dye compositions of the present invention, is (are) preferably present in a proportion of from 0.01% to 10% by. weight approximately and in particular in a proportion of from 0.1% to 5% by weight approximately relative to the total weight of the composition.

According to the present invention, the classes of thickening polymers that are more particularly preferred are chosen from classes (i), (ii), (iii), (v), (vii) and (viii) listed above.

The dye composition according to the invention may also contain one or more couplers other than the 3,5-diaminopyridines of formula (I) according to the invention and chosen from meta-phenylenediamines, meta-aminophenols, meta-diphenols, naphthols and heterocyclic couplers such as pyrazolo[1,5-b]-1,2,4-triazoles, pyrazolo[3,2-c]-1,2,4-triazoles, pyrazol-5-ones, pyridines other than the. 3,5-diaminopyridines of formula (I) according to the invention, indoles, indolines, indazoles, benzimidazoles, benzothiazoles, benzoxazoles, 1,3-benzodioxoles and quinolines, and the addition salts thereof with an acid.

These couplers are more particularly chosen from 2,4-diamino-1-(β-hydroxyethyloxy)benzene, 2-methyl-5-aminophenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2-amino-4-(β-hydroxyethylamino)-1-methoxybenzene, 1,3-diaminobenzene, 1,3-bis(2,4-diaminophenoxy)propane, sesamol, α-naphthol, 2-methyl-1-naphthol, 6-hydroxyindole, 4-hydroxyindole, 4-hydroxy-N-methylindole, 6-hydroxyindoline, 2, 6-dihydroxy-4-methylpyridine, 1-H-3-methylpyrazol-5-one, 1-phenyl-3-methylpyrazol-5-one, 2-amino-3-hydroxypyridine, 3,6-dimethylpyrazolo[3,2-c]-1,2,4-triazole, 2,6-dimethylpyrazolo[1,5-b]-1,2,4-triazole and 3-(4-hydroxy-1-methyl-1H-indol-5-ylmethyl)-1-methylpyridinium, and the addition salts thereof with an acid.

When they are present, the additional coupler(s) preferably represent(s) from 0.0001% to 10% by weight approximately relative to the total weight of the dye composition in accordance with the invention, and even more preferably from 0.005% to 5% by weight approximately relative to this weight.

The addition salts with an acid of the 3,5-diaminopyridines of formula (I), of the oxidation dye-precursors and of the additional couplers that may be present, and which may be used in the dye compositions according to the invention, are chosen especially from the hydrochlorides, hydrobromides, sulfates, tartrates, lactates and acetates.

The dye composition according to the invention may also contain direct dyes used especially to modify the shades by enriching them with glints; in this case, they may be chosen from neutral, cationic or anionic nitro dyes, azo dyes or anthraquinone dyes, in a weight proportion of from 0.001% to 20% approximately. and preferably from 0.01% to 10% relative to the total weight of the composition.

The medium for the composition that is suitable for dyeing is preferably an aqueous medium consisting of water and can advantageously contain cosmetically acceptable organic solvents including, more particularly, alcohols such as ethyl alcohol, isopropyl alcohol, benzyl alcohol and phenylethyl alcohol, or glycols or glycol ethers such as, for example, ethylene glycol monomethyl, monoethyl or monobutyl ether, propylene glycol or its ethers such as, for example, propylene glycol monomethyl ether, butylene glycol, dipropylene glycol, and also diethylene glycol alkyl ethers such as, for example, diethylene glycol monoethyl ether or monobutyl ether, in concentrations ranging from about 0.5% to about 20% and preferably between about 2% and 10% by weight relative to the total weight of the composition.

The composition according to the invention may also contain an effective amount of other agents, known previously elsewhere in oxidation dyeing, such as various common adjuvants, for instance sequestering agents such as EDTA and etidronic acid, UV screening agents, waxes, volatile or nonvolatile, cyclic or linear or branched silicones, preserving agents, ceramides, pseudoceramides, plant, mineral or synthetic oils, vitamins or provitamins, for instance panthenol, opacifiers, or polymers other than those of the invention.

The composition according to the invention preferably contains a cationic polymer.

Said composition may also contain reducing agents or antioxidants. These agents may be chosen in particular from sodium sulfite, thioglycolic acid, thiolactic acid, sodium bisulfite, dehydroascorbic acid, hydroquinqne, 2-methylhydroquinone, tert-butylhydroquinone and homogentisic acid, and, in this case, they are generally present in amounts ranging from about 0.05% to 1.5% by weight relative to the total weight of the composition.

The composition according to the invention can also contain one or more fatty alcohols, these fatty alcohols being introduced in pure form or as a mixture. Among these, mention may be made more particularly of lauryl alcohol, cetyl alcohol, stearyl alcohol and oleyl alcohol and mixtures thereof. These fatty alcohols can represent from 0.001% to 20% by weight approximately relative to the total weight of the composition.

Preferably, the dye composition and/or the oxidizing composition of the ready-to-use composition according to the invention contains at least one nonionic, anionic, cationic or amphoteric surfactant in a proportion of from 0.1% to 20% by weight approximately.

Even more preferably, said composition contains at least one nonionic surfactant.

Needless to say, a person skilled in-the art will take care to select the optional additional compound(s) mentioned above such that the advantageous properties intrinsically associated with the oxidation dye composition according to the invention are not, or are not substantially, adversely affected by the envisaged addition(s).

In the oxidizing composition, the oxidizing agent is preferably chosen from hydrogen peroxide, urea peroxide, alkali metal bromates or ferricyanides, and persalts such as perborates and persulfates. It is particularly preferred to use hydrogen peroxide. This oxidizing agent advantageously consists of an aqueous hydrogen peroxide solution whose titer may range, more particularly, from about 1 to 40 volumes and even more preferably from about 5 to 40 volumes.

Oxidizing agents that may also be used are one or more redox enzymes such as laccases, peroxidases and 2-electron oxidoreductases, where appropriate in the presence of the respective donor or cofactor thereof.

The pH of the dye composition or of the ready-to-use composition applied to the keratin fibers [composition resulting from mixing together the dye composition according to the invention and the oxidizing composition] is generally between 3 and 12. It is preferably between 6 and 11 and may be adjusted to the desired value using acidifying or basifying agents that are well known in the prior art in the dyeing of keratin fibers.

Among the basifying agents that may be mentioned, for example, are aqueous ammonia, alkali metal carbonates, alkanolamines such as monoethanolamine, diethanolamine and triethanolamine and derivatives thereof, oxyethylenated and/or oxypropylenated hydroxyalkylamines and ethylenediamines, sodium hydroxide, potassium hydroxide and the compounds of formula (VIII) below:

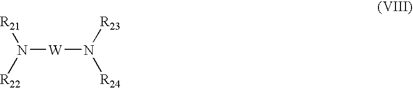

in which W is a propylene residue optionally substituted with a hydroxyl group or a C₁-C₄ alkyl radical; R₂₁, R₂₂, R₂₃ and R₂₄, which may be identical or different, represent a hydrogen atom, a C₁-C₄ alkyl radical or a C₁-C₄ hydroxyalkyl radical.

The acidifying agents are conventionally, for example, mineral or organic acids, for instance hydrochloric acid, orthophosphoric acid, carboxylic acids, for instance tartaric acid, citric acid or lactic acid, or sulfonic acids.

The dye composition in accordance with the invention may be in various forms, such as in the form of liquids, powders, creams or gels, that may be pressurized, or in any other form that is suitable for dyeing keratin fibers, and especially human hair.

The dyeing process according to the invention preferably consists in applying the ready-to-use composition, prepared extemporaneously at the time of use from the dye composition according to the invention and the oxidizing composition described above, to wet or dry keratin fibers, and in leaving the composition to act for an exposure time preferably ranging from 1 to 60 minutes approximately, and mote preferably from 10 to 45 minutes approximately, in rinsing the fibers and then in optionally washing them with shampoo, rinsing them again and then drying them.

The examples that follow are intended to illustrate the invention.

### EXAMPLES 1 to 3

The three dye composition below were prepared:

(expressed in grams - AM* denotes Active Material)

| | Example 1 | Example 2 | Example 3 |
|---|---|---|---|
| 2,6-Dimethoxy-3,5-diaminopyridine | 0.363 | 0.484 | 0.605 |
| dihydrochloride (coupler of | | | |
| formula (I)) | | | |
| para-Phenylenediamine | 0.324 | | |
| (oxidation base) | | | |
| N,N-Bis(β-hydroxyethyl)-para- | | | |
| phenylenediamine sulfate | | 0.936 | |
| (oxidation base) | | | |
| 2-(β-Hydroxyethyl)-para- | | | 1.125 |
| phenylenediamine | | | |
| dihydrochloride (oxidation base) | | | |
| 2,4-Diamino-1-(β-hydroxyethyloxy) | 0.361 | 0.241 | 0.602 |
| benzene dihydrochloride | | | |
| (additional coupler) | | | |
| Aculyn 44 sold by the company | 4 | | |
| Rohm & Haas (fatty-chain nonionic | | | |
| amphiphilic polymer) | | | |
| Carbopol 980 sold by the company | | 1 | |
| Goodrich (crosslinked | | | |
| polyacrylic acid) | | | |
| Jaguar HP105 sold by the company | | | 2 |
| Rhodia Chimie (hydroxypropyl guar) | | | |
| Common dye support | (*) | (*) | (*) |

| | |
|---|---|
| (*) Common dye support | |
| Sodium lauryl ether sulfate oxyethylenated with 2.2 | 6.44 AM* |
| mol of ethylene oxide, as an aqueous 70% solution, sold | |
| under the name Texapon NSW by the company Cognis | |
| (Linear 50% 70/30 C13/C15)alkyl ether carboxylic | 5.0 |
| acid monoethanolamide oxyethylenated with 2 mol of | |
| ethylene oxide | |
| Pentasodium salt of diethylenetriaminepentaacetic acid as | 0.80 AM* |
| an aqueous 40% solution, sold under the name Dissoluine | |
| D-40 ® by the company Akzo | |
| Ammonium thiolactate as an aqueous 58% solution | 0.46 AM* |
| Aqueous ammonia containing 20.5% NH₃ | 12.0 |
| Demineralized water | qs 100 |

At the time of use, each of the dye compositions described above was mixed weight-for-weight with a 20-volumes hydrogen peroxide solution (6% by weight) of pH 3.

The mixtures thus prepared were applied for 30 minutes to locks of permanent-waved natural gray hair containing 90% white hairs. The locks were then rinsed, washed with a standard shampoo, rinsed again and then dried. They were dyed in the shades collated in the table below:

| EXAMPLES | Shade |
|---|---|
| 1 | Ash natural brown |
| 2 | Ash natural chestnut |
| 3 | Ash natural brown |

1. A composition for the oxidation dyeing of keratin fibers, in particular of human keratin fibers such as the hair, comprising, in a medium that is suitable for dyeing:
(a) as coupler, at least one 3,5-diaminopyridine derivative of formula (I) below: 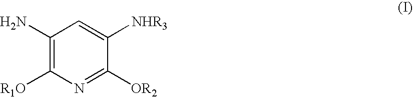
in which
R₁ and R₂, which may be identical or different, represent a C₁-C₄ alkyl, C₁-C₄ monohydroxyalkyl or C₂-C₄ polyhydroxyalkyl radical,
R₃ represents a hydrogen atom or a C₁-C₄ alkyl, C₁-C₄ monohydroxyalkyl or C₂-C₄ polyhydroxyalkyl radical, or one of the addition salts thereof with an acid; and
(b) at least one oxidation dye precursor, characterized in that it also comprises at least one thickening polymer chosen from the group consisting of:
(i) amphiphilic polymers comprising at least one fatty chain;
(ii) crosslinked acrylic acid homopolymers;
(iii) crosslinked copolymers of (meth)acrylic acid and of a (C₁-C₆)alkyl acrylate;
(iv) nonionic homopolymers and copolymers containing ethylenically unsaturated monomers of ester and/or amide type;
(v) crosslinked homopolymers of 2-acrylamido-2-methylpropanesulfonic acid and the partially or totally neutralized crosslinked acrylamide copolymers thereof;
(vi) ammonium acrylate homopolymers or copolymers of ammonium acrylate and of acrylamide;
(vii) nonionic guar gums;
(viii) biopolysaccharide gums of microbial origin, and in particular scleroglucan gum or xanthan gum;
(ix) gums derived from plant exudates, and in particular gum arabic, ghatti gum, karaya gum, gum tragacanth, carrageenan gum, agar gum and carob gum;
(x) hydroxypropylcelluloses or carboxyalkylcelluloses;
(xi) pectins;
(xii) alginates.

2. The composition as claimed in claim 1, characterized in that the 3,5-diaminopyridines of formula (I) are chosen from 2,6-dimethoxy-3,5-diaminopyridine, 2,6-diethoxy-3,5-diaminopyridine and 2,6-di(β-hydroxyethyloxy)-3,5-diaminopyridine, and the addition salts thereof with an acid.

3. The composition as claimed in claim 2, characterized in that the 3,5-diaminopyridine is 2,6-dimethoxy-3,5-diaminopyridine, or an addition salt thereof with an acid.

4. The composition as claimed in any one of the preceding claims, characterized in that the 3,5-diaminopyridines of formula (I) and the addition salts thereof with an acid represent from 0.0001% to 10% by weight relative to the total weight of the composition.

5. The composition as claimed in claim 4, characterized in that the 3,5-diaminopyridines of formula (I) and the addition salts thereof with an acid represent from 0.005% to 5% by weight relative to the total weight of the composition.

6. The composition as claimed in claim 1, characterized in that the oxidation dye precursor is chosen from para-phenylenediamines, double bases, para-aminophenols, ortho-aminophenols and heterocyclic. oxidation bases, and the addition salts thereof with an acid.

7. The composition as claimed in any one of the preceding claims, characterized in that the oxidation dye precursor(s) represent(s) from 0.0005% to 12% by weight relative to the total weight of the composition.

8. The composition as claimed in any one of the preceding claims, characterized in that the fatty-chain amphiphilic thickening polymer is chosen from those of anionic, nonionic or cationic type.

9. The composition as claimed in claim 8, characterized in that the fatty-chain amphiphilic thickening polymer is chosen from those of nonionic type.

10. The composition as claimed in any one of claims 1 to 7, characterized in that the thickening polymer is chosen from crosslinked acrylic acid homopolymers.

11. The composition as claimed in any one of claims 1 to 7, characterized in that the thickening polymer is chosen from crosslinked copolymers of (meth)acrylic acid and of a (C₁-C₆)alkyl acrylate.

12. The composition as claimed in any one of claims 1 to 7, characterized in that the thickening polymer is chosen from crosslinked 2-acrylamido-2-methylpropanesulfonic acid homopolymers and partially or totally neutralized crosslinked acrylamide copolymers thereof.

13. The composition as claimed in any one-of claims 1 to 7, characterized in that the thickening polymer is chosen from nonionic guar gums.

14. The composition as claimed in any one of claims 1 to 7, characterized in that the thickening polymer is chosen from scleroglucan gum and xanthan gum.

15. The composition as claimed in any one of the preceding claims, characterized in that the thickening polymer(s) represent(s) from 0.01% to 10% by weight relative to the total weight of the composition.

16. The composition as claimed in claim 15, characterized in that the thickening polymer(s) represent(s) from 0.1% to 5% by weight relative to the total weight of the composition.

17. The composition as claimed in any one of the preceding claims, characterized in that it contains at least one additional coupler.

18. The composition as claimed in claim 17, characterized in that the couplers are chosen from meta-phenylenediamines, meta-aminophenols, meta-diphenols, naphthols and heterocyclic couplers other than the 3,5-diaminopyridines of formula (I), and the addition salts of these compounds with an acid.

19. The composition as claimed in any one of the preceding claims, characterized in that the couplers are present in concentrations ranging from 0.0001% to 10% by weight relative to the total weight of the composition.

20. The composition as claimed in any one of the preceding claims, characterized in that the addition salts with an acid of the 3,5-diaminopyridines of formula (I), of the oxidation dye precursors and of the additional couplers are chosen from the hydrochlorides, hydrobromides, sulfates, tartrates, lactates and acetates.

21. The composition as claimed in any one of the preceding claims, characterized in that it also contains at least one direct dye in a proportion of from 0.001% to 20% by weight relative to the total weight of the composition.

22. The composition as claimed in any one of the preceding claims, characterized in that it also contains at least one reducing agent or antioxidant, in amounts ranging from 0.05% to 1.5% by weight relative to the total weight of the composition.

23. A ready-to-use composition for the oxidation dyeing of keratin fibers, in particular of human keratin fibers such as the hair, characterized in that it is obtained by mixing together a dye composition as defined in any one of claims 1 to 22 and an oxidizing composition containing at least one oxidizing agent, with the exclusion of the coupler 2,6-dimethoxy-3,5-diaminopyridine combined with the methacrylic acid/ethyl acrylate/stearyl methacrylate oxyethylenated terpolymer in a uricase-containing enzymatic oxidizing system.

24. The composition as claimed in claim 23, characterized in that the oxidizing agent is chosen from hydrogen peroxide, urea peroxide, alkali metal bromates or ferricyanides, persalts, redox enzymes such as laccases, peroxidases and 2-electron oxidoreductases, where appropriate in the presence of the respective donor or cofactor thereof.

25. The composition as claimed in claim 24, characterized in that the oxidizing agent is hydrogen peroxide.

26. The composition as claimed in claim 25, characterized in that the oxidizing agent is an aqueous hydrogen peroxide solution with a titer ranging from 1 to 40 volumes.

27. The composition as claimed in any one of the preceding claims, characterized in that it has a pH ranging from 3 to 12.

28. The composition as claimed in claim 23, characterized in that the dye composition and/or the oxidizing composition contains at least one surfactant chosen from anionic, cationic, nonionic and amphoteric surfactants in a proportion of from 0.1% to 20% by weight relative to the total weight of the composition.

29. A process for the oxidation dyeing of keratin fibers, and in particular of human keratin fibers such as the hair, which consists in applying to the fibers a dye composition containing, in a medium that is suitable for dyeing, at least one 3,5-diaminopyridine of formula (I) as defined in any one of claims 1 to 6, the color being developed at alkaline, neutral or acidic pH with the aid of an oxidizing composition that is mixed with the dye composition just at the time of use, or that is applied sequentially without intermediate rinsing, at least one thickening polymer as defined in any one of claims 1 and 9 to 14 being present in the dye composition and/or oxidizing composition.

30. A multi-compartment device or “kit” for the oxidation dyeing of keratin fibers, and in particular of human keratin fibers such as the hair, characterized in that it comprises at least two compartments, one of which contains at least one 3,5-diaminopyridine of formula (I) as defined in any one of claims 1 to 6, and a second compartment containing at least one oxidizing agent, at least one thickening polymer as defined in any one of claims 1 and 9 to 14 being present in the first compartment and/or in the second compartment.

31. A multi-compartment device or “kit” for the oxidation dyeing of keratin fibers, and in particular of human keratin fibers such as the hair, characterized in that it comprises at least one compartment containing at least one 3,5-diaminopyridine of formula (I) as defined in any one of claims 1 to 6, at least one compartment containing at least one thickening polymer as defined in any one of claims 1 and 9 to 14, and at least one other compartment containing at least one oxidizing agent.
